(19) United States
(12) Patent Application Publication
(10) Pub. No.: US 2025/0155424 A1
(43) Pub. Date: May 15, 2025

(54) METHODS FOR DETERMINING TOTAL ARSENIC, DISSOLVED ARSENIC, PARTICULATE ARSENIC, VOLATILE ARSENIC AND IONIC ARSENIC IN PETROLEUM, PETROLEUM DERIVATIVES AND SHALE OIL AND SET OF WASHING BOTTLES FOR USE IN THE METHODS

(71) Applicants: PETRÓLEO BRASILEIRO S.A. – PETROBRAS, Rio de Janeiro (BR); CENTRO DE TECNOLOGIA MINERAL - CETEM, Rio de Janeiro (BR)

(72) Inventors: Rainerio ESCALFONI JUNIOR, Rio de Janeiro (BR); Isabela Caldeira Leite LEOCADIO, Rio de Janeiro (BR); Marcus Vinicius Eiffle DUARTE, Rio de Janeiro (BR); Manuel Castro CARNEIRO, Rio de Janeiro (BR); Maria Inês Couto MONTEIRO, Rio de Janeiro (BR); Fernanda Veronesi Marinho PONTES, Rio de Janeiro (BR); Lívia Gonçalves Leida SOARES, Rio de Janeiro (BR); Raísa Rodrigues GIOIA, Rio de Janeiro (BR); Arnaldo Alcover NETO, Rio de Janeiro (BR)

(21) Appl. No.: 18/938,671

(22) Filed: Nov. 6, 2024

(30) Foreign Application Priority Data
Nov. 9, 2023 (BR) 1020230234836

(51) Int. Cl. G01N 33/28

(52) U.S. Cl. CPC G01N 33/2835; G01N 33/2823

(57) ABSTRACT

This disclosure proposes an embodiment of a set of coupled washing bottles to be used in the method of separation and quantification of the arsenic fractions AsP, AsD and AsV of the samples, as well as an embodiment of a method for the determination of total arsenic (AsT), dissolved arsenic (AsD), particulate arsenic (AsP), volatile arsenic (AsV) and ionic arsenic (AsI) in petroleum, shale oil and related samples.

### FIELD OF THE DISCLOSURE

This disclosure belongs to the technical field of hydro-refining and treatment, more specifically in the prior characterization of potentially problematic samples for refining involving loads whose total arsenic content and its forms are known and must be identified and quantified for possible removal.

### BACKGROUND OF THE DISCLOSURE

The chemical composition of petroleum and its physical properties have a great variation. In general, its composition consists of a mixture of hydrocarbons (paraffinic, naphthenic, and aromatic) with a certain amount of metals and metalloids. The most abundant metals in petroleum are nickel (Ni), vanadium (V) and iron (Fe), and are present in concentrations ranging from 10 ppm to 1000 ppm. Other elements, such as lead (Pb), barium (Ba), tin (Sn), silver (Ag), cobalt (Co), copper (Cu), molybdenum (Mo), titanium (Ti) and zinc (Zn) are present in concentrations ranging from 1 ppm to 50 ppm. In addition, mercury (Hg) and arsenic (As) are present in concentrations of the order of 10 to 200 ppb. The metals and metalloids present in petroleum often negatively influence the performance of the products and, also, the petroleum refining process. Arsenic (As) is one of these possible metalloids present in the composition of petroleum.

The presence of arsenic causes several damages to the petroleum industry, such as: (i) irreversible poisoning of the catalysts used in the refining processes; (ii) reduction of the thermal stability of petroleum-derived products due to its participation in oxidative reactions; (iii) corrosion during the refining process; and (iv) environmental pollution. Furthermore, arsenic compounds present high levels of toxicity and are easily absorbed both orally and by inhalation. Knowing the chemical form of arsenic present in the samples of interest contributes to understanding these effects and, consequently, to studying their mitigation.

Petroleum, shale oil and related samples (naphtha, gasoline, diesel oil, lubricating oil, produced water and final refinery effluent) may contain arsenic in different inorganic and organic forms. Arsenic is present in most shale oils in relatively high concentrations, which can reach up to 200 ppm, that is, a thousand times more concentrated than in petroleum. In Brazil, CONAMA (National Environmental Council) established a maximum level of 500 μg/1 of total arsenic in liquid effluents from industries. The successful conversion of shale oil into liquid fuels is hindered by the presence of this element since it is a potent poison for catalysts. Arsenic is found in a variety of chemical and physical forms (species) (CRAMER et al., 1988), with each species presenting different characteristics in terms of solubility, volatility, toxicity, and reactivity. Knowledge of arsenic species and forms is essential to predict potential adverse effects during refining, as well as to optimize technologies for its removal from shale oil products.

According to Fish (1983), the molecular forms of trace elements in fossil deposits are complex and consist of a variable proportion of inorganic, organometallic and pseudo-organometallic (non-covalent carbon-element bond) chemical species residing in non-specific sites within the carbonaceous matrix.

Some analytical methodologies have been proposed to quantify and/or remove these chemical species.

U.S. Pat. No. 10,241,013 discloses a system and method for in-line and automatic dilution of chemicals of interest for speciation and subsequent analysis by inductively coupled plasma mass spectrometry (ICP-MS).

By providing automatic and in-line dilution, chemicals can be speciated and analyzed in real time, rather than pre-diluting each sample and allowing samples to wait for an automatic sampler to remove the pre-diluted sample for speciation and analysis. The document focuses on the separation into specific chemical species and the automatic dilution of samples, usually aqueous. However, there is no mention of the “operational species” designated as in this disclosure.

CN 210376254 discloses methods for quantifying arsenic and mercury in different forms in a body of water. The preparation of samples in an oily matrix is an important part of the presented disclosure. Water samples, even saline ones, constitute simpler matrices and can be introduced directly or by dilution into the ICP-MS equipment.

The point in common between this document and the disclosure under study is only the use of ICP-MS for the detection of arsenic in different forms. However, the focus of the document is the equipment and its use and not the identification method.

CN 105758830 discloses a method for measuring total arsenic content by digesting marine products in stages using a microwave humidification method. Although the document describes a method for measuring total arsenic content and discloses a digestion step, the sample preparation, digestion and measuring apparatus are different when compared to the disclosure under study. In this document, in addition to the preparation referring to samples of different origins, the concern lies in the quantification of total arsenic and not in the “operational species” or even in specific species, as in this disclosure.

The document Final Course Work (TCC) by Raisa Gioia from 2016 presented a study on the operational speciation of As in petroleum (or crude oil). The aim of this work was to develop an analytical method for the determination and quantification of total As (AsT) in the in natura sample and its dissolved fraction (AsD), particulate fraction (AsP) and volatile fraction (AsV). A high pressure and temperature system (HPA-S) was used for sample decomposition/digestion and the inductively coupled plasma mass spectrometry (ICP-MS) technique was used for quantification.

The main difference is that this disclosure presents a methodology for operational speciation of As, that is, it groups different species into useful typologies for possible future treatments of the streams under study or helping to anticipate where the contaminant will end up within the oil chain. Thus, for example, the quantification of total As (AsT) in the in natura sample delimits general quantity of contamination, the particulate arsenic fraction (AsP) indicates what could be separated by filtration or centrifugation, the volatile fraction (AsV) indicates the quantity that can reach the light fractions of direct distillation of oil and the ratio between the dissolved fraction (AsD) and ionic fraction (AsI) would show how much As could be extracted from the stream under study.

The work by Gioia in 2016 aimed to develop an analytical method for the determination of total arsenic (AsT) in raw crude oil samples and in dissolved particulate and volatile fractions, using high pressure and temperature digestion (High Pressure Asher-HPA-S) and the inductively coupled plasma mass spectrometry (ICP-MS) technique. This disclosure uses a more appropriate apparatus, with a coupled system and different analytical methods for quantifying arsenic fractions.

### SUMMARY OF THE DISCLOSURE

This disclosure proposes a set of coupled washing bottles (A, B) to be used in the method of separation and quantification of arsenic fractions AsP, AsD and AsV of the samples, as well as a method for determining total arsenic, dissolved arsenic, particulate arsenic, volatile arsenic and ionic arsenic in petroleum, shale oil and petroleum cuts in the naphtha, kerosene, and diesel range. The method has the following steps:
quantifying AsT;
determining the mass fraction of arsenic species;
determining the mass fraction of AsP;
determining the mass fraction of AsD and AsI; and
determining the mass fraction of AsV.

### BRIEF DESCRIPTION OF THE FIGURES

FIG. 1 shows the glassware used in the separation of the arsenic forms developed according to an embodiment of this disclosure.

FIG. 2 shows the separation scheme of the arsenic forms according to an embodiment of this disclosure.

FIG. 3 shows the flow of the samples according to an embodiment of this disclosure.

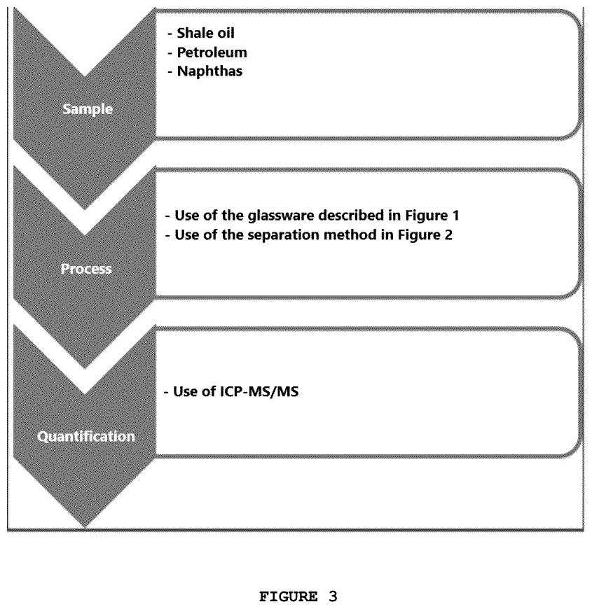

### DETAILED DESCRIPTION OF THE DISCLOSURE

This disclosure discloses a method for quantifying the forms of arsenic defined as: total (AsT), dissolved (AsD), particulate (AsP), volatile (AsV) and ionic (AsI) present in the dissolved fraction in petroleum samples, shale oil and related samples. The use of a sensitive, precise, and accurate analytical method will allow the adequate development of arsenic removal processes.

Volatile Arsenic (AsV) is the one that, at room temperature, is in the vapor phase and can be exhausted from the sample by bubbling gas.

Particulate Arsenic (AsP) is the one that, at room temperature, is in the solid phase and can be separated by filtration.

Dissolved Arsenic (AsD) is the one that, at room temperature, is in solution. It can be an organic or inorganic species.

Ionic Arsenic (AsI) is a type of AsD. It is a species of arsenic in the form of an ion.

Knowledge of the species and forms of arsenic is essential to predict potential adverse effects during refining, as well as to optimize technologies for its removal from shale oil products or in petroleum and petroleum derivatives.

### Glassware Used

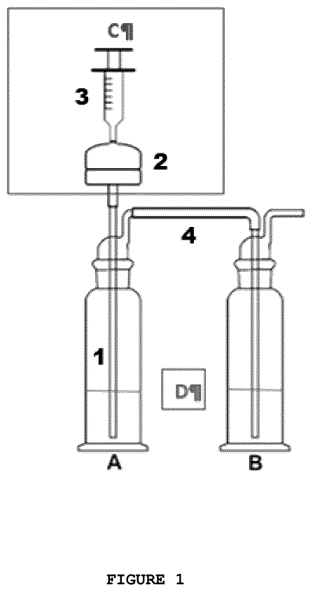

A set of coupled washing bottles (A, B) with a capacity of 50 ml and a sintered glass filter at the end of the central tube, connected by a rubber hose (4), was used to separate the fractions. A 25 mm diameter Swinnex support (1) (SX0002500) was attached to the bottle named A, where a 0.45 μm nylon membrane filter (HNWP02500) with a diameter of 25 mm (FIG. 1) was inserted.

Before inserting the sample, 10.00 ml of a saturated KBr solution in concentrated nitric acid (D) were added to each bottle (A, B).

It is worth noting that Gioia uses distinct and uncoupled systems to separate the arsenic fractions AsP, AsD and AsV of the samples. The use of a coupled system favors the speed of the process, using a smaller quantity of materials and samples. For the extraction of AsV, the TCC promotes the dragging of the fraction by using a vacuum pump inserted in Tube B, for 30 minutes, a different method from that used in this disclosure, which uses argon flow and a time 6 times shorter (5 minutes). In addition, for the extraction of AsP, the TCC uses a 5.0 μm Teflon filtration membrane while this disclosure uses a 0.45 μm Nylon membrane filter.

### Procedures

### Quantification of Total Arsenic (AsT)—Route I

Approximately 0.05 g of a sample of petroleum, petroleum derivatives (naphtha, gasoline, diesel oil or lubricating oil) or shale oil (+0.0001 g) were weighed in gelatin capsules and inserted into quartz tubes, where 2.00 ml of very pure HNO₃≥65% Sigma-Aldrich (CAS 84378) were added. The same procedure was adopted for the blank samples, which consisted of empty capsules. After digestion, the samples were made up to 15 ml with Milli-Q water with a resistivity of 18.2 MΩ.cm. AsT was then quantified via inductively coupled plasma mass spectrometer (ICP-MS) or triple quadrupole inductively coupled plasma mass spectrometer (ICP-MS/MS).

### Mass Quantification of Arsenic Species—Route II

The procedure for separation and mass quantification covered the following arsenic species: Volatile Arsenic (AsV), Particulate Arsenic (AsP), Dissolved Arsenic (AsD) and Ionic Arsenic (AsI).

Using a syringe (3), approximately 5 ml of a sample of petroleum, petroleum derivatives (naphtha, gasoline, diesel oil or lubricating oil) or shale oil were collected, the mass of which was measured, and then inserted into the support (1) containing the membrane filter (2) (assembly C) which, after filtration, is collected in bottle A.

After filtration, an argon flow (approximately 1 ml/min) is inserted into the inlet of the support (1), and maintained in the system for 5 minutes to collect the vapors in bottle B.

Thus, the AsP fraction was collected in the membrane filter (2), the AsD and AsI fractions in bottle A, after filtration, and the AsV fraction in bottle B, after the argon flow has passed.

With the exception of the AsV fraction, all fractions obtained were subjected to the acid digestion process in HPA-S according to the schedule described in Table 1.

### Determination of the Mass Fraction of AsP

The filter (2) containing the AsP fraction was left to dry at room temperature until it reached a constant weight. After its mass was determined, the filter (2) was inserted directly into the quartz tube of the HPA-S where 2.00 ml of very pure HNO₃≥65% Sigma-Aldrich (CAS 84378) was added and subjected to the digestion process. After digestion, the samples were made up to 15 ml with Milli-Q water with a resistivity of 18.2 MΩ.cm.

### Determination of the Mass Fraction of AsD and AsI

The sample filtered in bottle A containing the AsD and AsI fractions (AsD+I) had an aliquot removed and weighed inside a gelatin capsule. The capsule was then inserted directly into an HPA-S quartz tube where 2.00 ml of very pure HNO₃≥65% Sigma-Aldrich (CAS 84378) was added and subjected to the digestion process. After digestion, the samples were made up to 15 ml with Milli-Q water with a resistivity of 18.2 MΩ.cm.

To extract the ionic fraction (AsI), another aliquot (approximately 2 ml) of this fraction contained in bottle A was collected in a syringe, its mass was weighed and transferred to a falcon TPP tube, where 2.00 ml of NaOH 0.10 molar was added. The tube was vigorously shaken manually and left to rest for approximately 1 hour. Then, an aliquot of the aqueous fraction (lower) was partially collected and weighed in a gelatin capsule, and inserted into the HPA-S quartz tube, where 2.00 ml of very pure HNO₃≥65% Sigma-Aldrich (CAS 84378) was added and subjected to the digestion process. After digestion, the samples were made up to 15 ml with Milli-Q water with a resistivity of 18.2 MΩ.cm.

The determination of the mass fraction of AsD will be made by the difference obtained between these two procedures, according to the following equation:

$$AsD = AsD + I - AsI$$

### Determination of the Mass Fraction of AsV

The volatile fraction (AsV) that was collected in bottle B was made up to 100.00 ml with Milli-Q water, being analyzed directly in the ICP-MS/MS, without going through the digestion procedure.

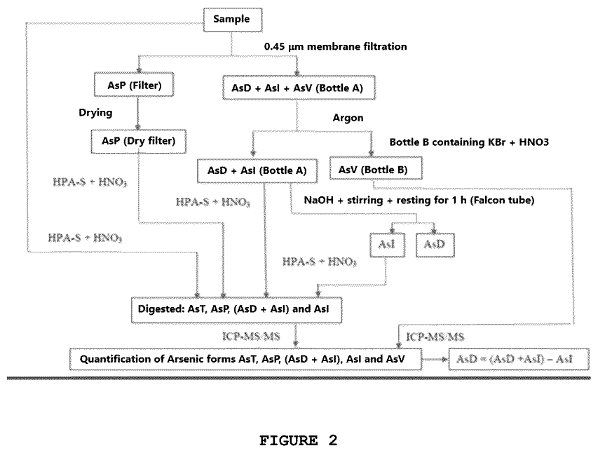

The separation scheme of the arsenic forms of this disclosure is shown in FIG. 2.

### Instrumental

### Analyses performed

An Anton Paar HPA-S high pressure and temperature digestion system (No. 80542445) with a quartz tube kit (14×15 ml tubes or 6×50 ml tubes) was used for digestion (or mineralization) of the samples. The operating conditions of the HPA-S are shown in Table 1.

TABLE 1
Operating conditions of the HPA-S

| Number of segments | (3) Time, Dwell, End |
|---|---|
| Number of cycles | 1 |
| Final temperature (° C.) | 280 |
| Heating ramp (Time, min) | 60 |
| Dwell (min) | 60 |

A triple quadrupole ICP-MS/MS model 8900(G3665A) mass spectrometer was used for arsenic quantification after sample pretreatment involving digestion. The ICP-MS/MS operating conditions are presented in Table 2. Both kinetic energy discrimination (use of the He collision cell) and the use of O₂ as the reaction gas were used to reduce possible polyatomic interferences and/or double load in arsenic measurements by ICP-MS/MS.

TABLE 2
ICP-MS/MS 8900 operating conditions (Agilent)

| | |
|---|---|
| Plasma carrier gas flow rate Ar (l/min) | 1.05 |
| Plasma gas (l/min) | 15.0 |
| Auxiliary gas (l/min) | 0.9 |
| Sampling depth (mm) | 10 |
| Plasma power (W) | 1550 |
| Nebulizer Type | Micromist |
| Autosampler Type | SPS4 |
| Ionic lens model | x-Lens |
| Reaction gas flow rate, ml/min (O₂) | 0.30 |
| Collision gas flow rate, ml/min (He) | 5.5 |
| On-mass monitored isotopes (He e O₂) | ⁷⁵As |
| Mass-shifted monitored isotopes (O₂) | ⁹¹AsO |
| Mode of operation | He e O₂ |

The use of triple quadruple equipment and collision/reaction gas are important for the determination of As in complex matrices, as they resolve interferences caused by polyatomic ions (such as ⁴⁰Ar³⁵Cl⁺, ³⁸Ar³⁷Cl⁺, ⁴⁰Ca³⁵Cl⁺, ⁵⁹Co¹⁶O⁺, and ⁵⁸Fe¹⁶OH⁺) and doubly loaded isotopes (such as ¹⁵⁰Nd²⁺ and ¹⁵⁰Sm²⁺). The use of collision gas promotes the formation of the species ⁹¹AsO⁺, which was also monitored in the analyses in this disclosure.

### EXAMPLES

Quantification of arsenic forms in shale oil samples and their distilled cuts/fractions (86/load, 86/11,86/residue, 61/load and 61/11 and 61/residue), petroleum naphtha (TES08090)

Table 3 shows the designation of the samples tested.

TABLE 3
Shale oil samples and fractions and petroleum naphtha studied.

| Identification | Description |
|---|---|
| 86/residue | Distilled fraction of the load >168° C. |
| | (Gas oil/diesel oil equivalent cut) |
| 61/residue | Distilled fraction of the load >188.6° C. |
| | (Gas oil/diesel oil equivalent cut) |
| 86/load | Distillation load (Shale Oil 86) |
| 61/load | Distillation load (Shale Oil 61) |
| 86/11 | Distilled fraction between 157.9° C. and |
| | 168.6° C. (naphtha/gasoline equivalent cut) |
| 61/11 | Distilled fraction between 169.4° C. and |
| | 188.6° C. (naphtha/gasoline equivalent cut) |
| TES08090 | Petroleum naphtha from catalytic cracking |

Tables 4, 5 and 6 present the results of AsT and its forms (AsP, AsD, AsI and AsV) for the tested samples. Recoveries of ASTₑₓₚₑᵣᵢₘₑₙₜₐₗ were calculated considering as 100% the mass fraction of AsT obtained by the sum of the forms (AsV+AsP+AsI+AsD). Recoveries between 75 and 125% were considered satisfactory. All recoveries of AST_{experimental (1)}, whose results were obtained at the same time as the quantification of arsenic forms, were satisfactory.

The arsenic fractions in most of the analyzed cuts of the samples are distributed in increasing order: AsV<AsP<AsI<AsD. An exception occurred for sample 61/load (1105 and 1089 ng/g for the AsP and AsI fractions, respectively).

TABLE 4
Results of quantification of total arsenic and its forms in distillation cuts of shale oil samples 86

| | 86/residue | | | 86/load | | | 86/11 | | |
|---|---|---|---|---|---|---|---|---|---|
| | | | Abundance/ | | | Abundance/ | | | Abundance/ |
| | Average | s | recovery | Average | s | recovery | Average | s | recovery |
| Form | (ng g⁻¹) | (ng g⁻¹) | (%)* | (ng g⁻¹) | (ng g⁻¹) | (%)* | (ng g⁻¹) | (ng g⁻¹) | (%)* |
| AsV | 3 | 1 | 0 | 8 | 2 | 0 | 12 | 9 | 6 |
| AsP | 721 | 164 | 3 | 752 | 100 | 10 | 30 | 4 | 14 |
| AsI | 2240 | 92 | 9 | 956 | 33 | 12 | 59 | 9 | 27 |
| AsD | 21261 | 5475 | 88 | 6190 | 388 | 78 | 119 | 2 | 54 |
| AsTₛᵤₘ | 24225 | 5478 | 100 | 7900 | 402 | 100 | 220 | 13 | 100 |
| AsT | 19537 | 1394 | 81 | 7676 | 243 | 97 | 217 | 20 | 99 |

TABLE 5
Results of quantification of total arsenic and its forms in distillation cuts of shale oil samples 61

| | 61/residue | | | 61/load | | | 61/11 | | |
|---|---|---|---|---|---|---|---|---|---|
| | | | Abundance/ | | | Abundance/ | | | Abundance/ |
| | Average | s | recovery | Average | s | recovery | Average | s | recovery |
| Form | (ng g⁻¹) | (ng g⁻¹) | (%)* | (ng g⁻¹) | (ng g⁻¹) | (%)* | (ng g⁻¹) | (ng g⁻¹) | (%)* |
| AsV | 5 | 1 | 0.01 | 3 | 1 | 0.02 | 1 | 1 | 0 |
| AsP | 2825 | 603 | 5 | 1105 | 115 | 8 | 74 | 2 | 12 |
| AsI | 11717 | 653 | 19 | 1089 | 56 | 8 | 120 | 12 | 19 |
| AsD | 45840 | 1019 | 76 | 12129 | 312 | 85 | 422 | 11 | 68 |
| AsTₛᵤₘ | 60387 | 1352 | 100 | 14326 | 338 | 100 | 616 | 17 | 100 |
| AsT | 68746 | 3181 | 114 | 13630 | 292 | 95 | 702 | 42 | 114 |

*Abundance/Recovery—abundance of arsenic forms relative to the ASTₛᵤₘ content or recovery of AsT relative to the AsTₛᵤₘ content;
s—standard deviation;
AsV—volatile arsenic;
AsP—particulate arsenic;
AsI—ionic arsenic;
AsD—dissolved arsenic;
ASTₛᵤₘ—total arsenic calculated by the sum of arsenic forms;
AST—total arsenic quantified at the same time as the quantification of arsenic forms.

TABLE 6
Results of the quantification of total arsenic and
its forms in the petroleum naphtha sample TES08090

| | TES08091 | | |
|---|---|---|---|
| | Average | s | Abundance/recovery |
| Form | (ng g⁻¹) | (ng g⁻¹) | (%) |
| AsV | 1.2 | 0.2 | 2.8 |
| AsD + I | 41 | 4 | 97.2 |
| AsTₛᵤₘ | 42.2 | — | 100 |
| AsTₑₓₚₑᵣᵢₘₑₙₜₐₗ | 46 | 9 | 109 |

1. A method for determining total arsenic (AsT), dissolved arsenic (AsD), particulate arsenic (AsP), volatile arsenic (AsV) and ionic arsenic (AsI) in petroleum, petroleum derivatives and shale oil, the method comprising:
quantifying AsT;
determining the mass fraction of arsenic species;
determining the mass fraction of AsP;
determining the mass fraction of AsD and AsI; and
determining the mass fraction of AsV.

2. A method according to claim 1, wherein the quantifying AsT comprises:
weighing a sample of petroleum, petroleum derivatives or shale oil,
digesting the sample with concentrated and sub-distilled HNO₃ -in a high pressure and temperature system (HPA-S),
after digestion, transferring the resulting solution to a tube with Milli-Q type water, and
quantifying AsT via inductively coupled plasma mass spectrometer (ICP-MS) or triple quadrupole inductively coupled plasma mass spectrometer (ICP-MS/MS), and
wherein the petroleum derivatives include one or more of naphtha, gasoline, diesel oil, or lubricating oil.

3. A method according to claim 2, wherein the determining the mass fraction of Arsenic species comprises:
collecting with a syringe the sample of petroleum, petroleum derivatives, or shale oil to be tested, whose mass was measured, and then inserting it into a support containing a membrane filter which, after filtration, is collected in a first bottle,
after filtration, maintaining an argon flow in the system, inserted through the support for a selected period of time, and
collecting the vapors in second bottle.

4. A method according to claim 3, wherein the AsP fraction is collected in the membrane filter, the AsD and AsI fractions are collected in a first flask, and the AsV fraction is collected in second flask after the argon flow has passed.

5. A method according to claim 4, wherein with the exception of the AsV fraction, all the fractions obtained are subjected to the high pressure and temperature acid digestion process (HPA-S).

6. A method according to claim 1, wherein the determining the mass fraction of AsP comprises:
allowing a filter containing the AsP fraction to dry at room temperature until it reaches a constant weight to determine its mass,
after its mass has been determined, inserting the filter directly into the HPA-S quartz tube,
adding a selected volume of concentrated HNO₃, and
subjecting the selected volume to the digestion process.

7. A method according to claim 1, wherein the determining the mass fraction of AsD comprises:
removing an aliquot of the filtered sample in the bottle (A) having the fractions AsD and AsI (AsD+I),
weighing said aliquot removed inside a gelatin capsule, and
inserting the capsule directly into a quartz tube to be digested in the HPA-S.

8. A method according to claim 2, wherein the determining the mass fraction of AsI comprises:
removing another aliquot of the fraction contained in the first bottle containing the fractions AsD and AsI (AsD+I),
weighing the aliquot removed,
transferring to a TPP falcon tube,
adding a selected volume of NaOH 0.10 molar,
manually and vigorously shaking the tube with the aliquot,
after shaking, letting the tube with the aliquot rest for 1 hour,
after resting, collecting the aliquot of the aqueous fraction,
weighing it in a gelatin capsule,
inserting it into the HPA-S quartz tube and adding 2.00 ml of concentrated HNO₃ and subjecting it to the digestion process, and
wherein the mass fraction of AsD is calculated by the difference obtained according to the following equation:
$$AsD = AsD + I - AsI .$$

9. A method according to claim 8, wherein the determining the mass fraction of AsV comprises:
collecting the volatile fraction (AsV) in the second bottle (B),
filling the second bottle with Milli-Q water, and
analyzing directly in the ICP-MS/MS.

10. A method according to claim 9, wherein the determining the mass fraction of AsV further does not involve the digestion procedure.

11. A set of washing bottles for use in the method as defined in claim 10, wherein each of the first bottle and the second bottle is coupled by a rubber hose, wherein the first bottle also includes the support with the membrane filter, and wherein the first and second bottles each are coupled with a syringe when an aliquot of sample is inserted into the first and second bottles.

12. The set according to claim 11, wherein the membrane filter comprises 0.45 μm pore nylon.
